(19) United States
(12) Patent Application Publication
(10) Pub. No.: US 2021/0169897 A1
(43) Pub. Date: Jun. 10, 2021

(54) Concentrated Fulvestrant Compositions

(71) Applicant: Nevakar Inc., Bridgewater, NJ (US)

(72) Inventors: Vivek Yadav, Henderson, NV (US); Sriramya Garapati, North Brunswick, NJ (US); Bronislava Zektser, Bedminster, NJ (US); Varun Khurana, Raritan, NJ (US); Iouri V Ilitchev, Hillsborough, NJ (US); Prem Sagar Akasapu, Edison, NJ (US); Tushar Hingorani, Bridgewater, NJ (US); Kumaresh Soppimath, Skillman, NJ (US); Navneet Puri, Lebanon, NJ (US)

(21) Appl. No.: 16/769,204

(22) PCT Filed: Dec. 6, 2018
(86) PCT No.: PCT/US2018/064319

§ 371 Date: Jun. 2, 2020

Related U.S. Application Data
(60) Provisional application No. 62/595,938, filed on Dec. 7, 2017.

(51) Int. Cl. A61K 31/565; A61K 9/00; A61K 9/08; A61K 47/12; A61K 47/14; A61K 47/44

(52) U.S. Cl. CPC A61K 31/565; A61K 9/0019; A61K 47/44; A61K 47/12; A61K 47/14; A61K 9/08

(57) ABSTRACT

Compositions and methods for injectable and liquid fulvestrant formulations that have fulvestrant at a relatively high concentration stably solubilized before and after injection of the patient are provided.

This application claims priority to our copending U.S. Provisional Application with the Ser. No. 62/595,938, which was filed Dec. 7, 2017.

### FIELD OF THE INVENTION

The field of the invention is pharmaceutical compositions containing fulvestrant at high concentrations which can be injected intramuscularly.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Fulvestrant, a known estrogen receptor antagonist, is currently approved for use in the treatment of hormone positive breast cancer. Faslodex® (commercially available Fulvestrant formulation) consists of fulvestrant 50 mg/mL, 10% w/v ethanol, 10% w/v benzyl alcohol, 15% w/v benzyl benzoate, made up to 100% with castor oil. Fulvestrant is administered to the patient intramuscularly. Since the solubility of fulvestrant in the above mentioned solvent is limited, administration is often challenging. As the required dose of fulvestrant for a patient is generally 500 mg, Faslodex® is administered to the patient as two 5 mL injections, one in each buttock on days 1, 15, 29 and then monthly thereafter. Due to the relatively high viscosity and large volume administered, a number of injection site reactions have been reported.

Fulvestrant shows relatively high solubility in non-aqueous solvents such as ethanol (>200 mg/mL) and benzyl alcohol (>200 mg/mL) which would reduce administration volume to less than 3 mL. However, such solvents cannot be used for administration as fulvestrant will precipitate in vivo, which may cause injection site reactions and leads to inconsistent pharmacokinetics. Thus, despite high concentration of fulvestrant can be achieved in certain solvents or mixture of solvents, preventing precipitation of fulvestrant when administered in vivo remains a significant challenge.

Thus, there remains a need for improved compositions for injectable fulvestrant formulation at high concentration.

### SUMMARY OF THE INVENTION

The inventive subject matter is directed to various compositions of and methods for injectable, liquid fulvestrant formulations having fulvestrant solubilized before the injection at a relatively high concentration into a patient, where the formulation prevents precipitation of fulvestrant. Consequently, the volume of fulvestrant injection per dose can be significantly substantially reduced.

In one aspect of the inventive subject matter, the inventors contemplate an injectable liquid pharmaceutical composition that comprises fulvestrant dissolved in a liquid solvent at a concentration of at least 60 mg/ml, wherein the liquid solvent is a pharmaceutically acceptable carrier for injection. In especially preferred aspects, the liquid solvent comprises a free ricinoleic acid and/or hydrolyzed castor oil, and may further include ethanol and/or benzyl alcohol. For example, the free ricinoleic acid or hydrolyzed castor oil may be present at a concentration of at least 40 or 50% w/v, while ethanol may be present at a concentration of between 5-25% w/v, and/or benzyl alcohol may be present at a concentration of between 5-25% w/v.

In further contemplated aspects, fulvestrant may be dissolved in the liquid solvent at a concentration of at least 80 mg/ml, and the composition will have a viscosity of equal or less than 100 cP. Moreover, it is generally preferred that the solubility of fulvestrant in the liquid solvent is maintained at least for 48 hours within an in vitro precipitation model. Among other embodiments, contemplated compositions may also comprise oleic acid, wherein the oleic acid is present at a concentration less than 25%, and/or further comprise benzyl benzoate, wherein the benzyl benzoate is present at a concentration at or less than 15%. Therefore, suitable compositions may include ethanol, benzyl alcohol, oleic acid, and ricinoleic acid or hydrolyzed castor oil, and optionally further comprise benzyl benzoate. In other embodiments, the liquid solvent may comprise 10% ethanol, 10% benzyl alcohol, 15% oleic acid, and q.s. ricinoleic acid or hydrolyzed castor oil. Preferably, injectable liquid pharmaceutical compositions are formulated in a volume equal to or less than 5 ml to provide a therapeutically effective dose to the patient.

In another aspect of the inventive subject matter, the inventors contemplate an injectable liquid pharmaceutical composition that includes fulvestrant dissolved in a liquid solvent that maintains a viscosity at less than 100 Cp, wherein the solvent prevents precipitation of the fulvestrant after injection into a person. For example, fulvestrant may be dissolved in the liquid solvent at a concentration of at least 60, or at least 70, or at least 80 mg/ml. While not limiting the inventive subject matter, it is preferred that the liquid solvent comprises free ricinoleic acid (i.e., not esterified with glycerol) or hydrolyzed castor oil, ethanol, benzyl alcohol, and optionally benzyl benzoate. Preferably, the free ricinoleic acid or hydrolyzed castor oil is present at a concentration of at least 50% w/v, the ethanol is present at a concentration of between 5-25% w/v, and/or the benzyl alcohol is present at a concentration of between 5-25 w/v %. In further preferred aspects, fulvestrant is dissolved in the liquid solvent at a concentration of at least 80 mg/ml and the composition will have a viscosity of equal or less than 100 cP. As noted before, it is preferred that the solubility of fulvestrant in the liquid solvent is maintained at least for 48 hours before the injection in a gelatin in vitro test, and that solubility of the fulvestrant in the liquid solvent is maintained at least for 15 minutes after injection in vivo.

Additionally, or alternatively, contemplated compositions may comprise oleic acid, wherein the oleic acid is present at a concentration less than 25%, and/or may comprise benzyl benzoate, wherein the benzyl benzoate is present at a concentration at or less than 15%.

Viewed from a different perspective, contemplated injectable pharmaceutical compositions may therefore comprise a lipophilic compound dissolved in a liquid solvent, wherein the liquid solvent comprises free ricinoleic acid or hydrolyzed castor oil. Most typically, but not necessarily, the lipophilic compound is fulvestrant, and/or the liquid solvent comprises ethanol and benzyl alcohol. In such compositions, it is contemplated that the free ricinoleic acid is present at a concentration of at least 40, or at least 50% (w/v), that ethanol is present at a concentration of between 5-25% (w/v), and/or that benzyl alcohol is present at a concentration of between 5-25% (w/v). Most typically, solubility of the lipophilic compound in the liquid solvent is maintained at least for 48 hours at room temperature, or solubility of the lipophilic compound in the liquid solvent is maintained at at least more than 80% of the maximum solubility level for 48 hours at a room temperature. It is further contemplated that the lipophilic compound has a p-coefficient of at least 3 in a 1-octanol/water system.

Therefore, in yet another aspect of the inventive subject matter, the inventors contemplate an injectable liquid pharmaceutical composition that comprises fulvestrant dissolved in a liquid solvent at a concentration of at least 60 mg/ml, wherein the liquid solvent comprises hydrolyzed castor oil. Where desired, the liquid solvent may further comprise ethanol and/or benzyl alcohol. Typically, the hydrolyzed castor oil is present at a concentration of at least 50% w/v, ethanol is present at a concentration of between 5-25% w/v, and/or benzyl alcohol is present at a concentration of between 5-25% w/v.

In such formulations, it is contemplated that the fulvestrant is dissolved in the liquid solvent at a concentration of at least 80 mg/ml. For example, fulvestrant may be dissolved in the liquid solvent at a concentration of at least 80 mg/ml wherein the injectable liquid pharmaceutical composition has a viscosity of less than 100 cP. Preferably, solubility of the fulvestrant in the liquid solvent is maintained at least for 48 hours within an in vitro precipitation model.

In further embodiments, contemplated composition may further include oleic acid, wherein the oleic acid is present at a concentration less than 25%, and/or further comprise benzyl benzoate, wherein the benzyl benzoate is present at a concentration at or less than 15%. Thus, suitable compositions will comprise ethanol, benzyl alcohol, oleic acid, and optionally further comprise benzyl benzoate. For example, contemplated compositions include those in which the liquid solvent comprises 10% ethanol, 10% benzyl alcohol, 15% oleic acid, and q.s. hydrolyzed castor oil, and optionally wherein a single dose of the injectable liquid pharmaceutical composition is formulated in a volume equal to or less than 5 ml.

Various objects, features, aspects and advantages of the inventive subject matter will become more apparent from the following detailed description of preferred embodiments.

### DETAILED DESCRIPTION

The inventors have surprisingly discovered that when fulvestrant is formulated in a solvent that comprises free ricinoleic acid (i.e., ricinoleic acid that is not esterified with an alcohol or polyol such as glycerol), fulvestrant can be formulated at a concentration of about 100 mg/mL without precipitation, even after injection into a patient's body. Such discovery is particularly unexpected as castor oil is a triglyceride where ricinoleic acid is the predominant fraction of fatty acids esterified with glycerol, and known castor oil-based formulations have a significantly limited ability to solubilize fulvestrant. It should be noted that as used herein, the term “free ricinoleic acid” refers to ricinoleic acid ((9Z,12R)-12-Hydroxyoctadec-9-enoic acid; CAS Number 141-22-0) that is not esterified with an alcohol or polyol such as glycerol. Moreover, it is further noted that the free ricinoleic acid may be synthetic, isolated and/or in at least partially purified form (e.g., from hydrolyzed castor oil or other hydrolyzed ricinoleic acid ester), or may even refer to ricinoleic acid provided as a crude castor oil hydrolysate.

Viewed from a different perspective, the inventors discovered that free ricinoleic acid can be provided as an isolated and/or purified ricinoleic acid, or can be provided in form of hydrolyzed castor oil providing the same surprising increase in solubility and stability. Moreover, the inventors unexpectedly found that where hydrolyzed castor oil was used as a source of ricinoleic acid, thusly produced formulations exhibited significantly reduced inflammation at the injection site. Regardless of the particular source of the ricinoleic acid, the inventors also discovered that the compositions contemplated herein advantageously and significantly reduced the viscosity of the pharmaceutical composition, which is typically associated with adverse injection site reactions and patient discomfort.

Additionally, the inventors also discovered that free ricinoleic acid can be used as a solvent for various lipophilic compounds other than fulvestrant (e.g., steroid drugs, various statins, cyclosporine, ketoprofen, itroconazole, carvedilol, etc.) to thereby provide various pharmaceutical compositions that provided high stability and high solubility of the lipophilic compound.

In an exemplary and preferred aspect of the subject matter, the inventors contemplate an injectable liquid pharmaceutical composition including fulvestrant in a pharmaceutically acceptable carrier. Most preferably, fulvestrant is dissolved in the pharmaceutically acceptable carrier at a concentration of at least 60 mg/ml, preferably at least 70 mg/ml, more preferably at least 80 mg/ml, and most preferably at least 100 mg/ml.

With respect to suitable pharmaceutically acceptable carrier, the inventors contemplate any aqueous, or non-aqueous carrier that can solubilize fulvestrant without significant toxicity to the patient when administered by injection (e.g., biocompatible). In addition, it is preferred that that the pharmaceutically acceptable carrier can dissolve fulvestrant without producing any significant impurities or side products, and/or will allow for a relatively low-viscosity formulation. Thus, in preferred embodiments, pharmaceutically acceptable carriers can dissolve fulvestrant at a concentration of at least 60 mg/ml (preferably at least 70 mg/ml, at least 80 mg/ml, at least 90 mg/ml, more preferably at least 100 mg/ml) with a viscosity of less than 200 cP, preferably less than 100 cP, more preferably less than 80 cP. Unless noted otherwise, the term “viscosity” as used herein refers to dynamic viscosity. Advantageously, use of free ricinoleic acid, or hydrolysis of castor oil to produce at least some free ricinoleic acid, will reduce viscosity of the formulation, and with that increases patient comfort and reduces precipitation of fulvestrant, while at the same time solubility is significantly increased in many formulations, especially where combined with at least one further co-solvent.

It is further preferred that suitable pharmaceutically acceptable carriers can dissolve fulvestrant at a concentration of at least 60 mg/ml (preferably at least 70 mg/ml, at least 80 mg/ml, at least 90 mg/ml, at least 100 mg/ml) to form a formulation and maintain the stability of the formulation at least 24 hours, preferably at least 48 hours, and more preferably at least 72 hours (e.g., measured by gelatin block assay), without producing any precipitant amount of more than 10%, preferably more than 5%, more preferably more than 3% of the previously dissolved fulvestrant in the formulation. It should be appreciated that the precipitation can be measured in various physical conditions, for example, preferably at a temperature between 15-40° C., between 15-35° C., between 15-25° C., or between 20-25° C. Thus, in other words, preferred pharmaceutically acceptable carriers can dissolve fulvestrant at a concentration of at least 60 mg/ml (at least 70 mg/ml, at least 80 mg/ml, at least 90 mg/ml, at least 100 mg/ml) to form a formulation that can be stable in vitro (room temperature (20° C.) for storage, before injection) for at least 24 hours, preferably at least 48 hours, and more preferably at least 72 hours, and/or in vivo (e.g., body temperature of the patient's body after injection) for at least 15 min, preferably at least 30 min, and more preferably at least 1 hour, most preferably at least 6 hours after injection.

In one especially preferred embodiment, the pharmaceutically acceptable carrier is a liquid solvent that includes monohydroxylated fatty acids, for example, free ricinoleic acid (single chain ricinoleic acid, 12-Hydroxy-cis-9-octadecenoic acid; (R,Z)-12-Hydroxyoctadec-9-enoic acid; C₁₈H₃₄O₃) or lesquerolic acid. In this context, it must be appreciated that free ricinoleic acid (i.e., free acid that is not esterified with an alcohol or polyol) is distinguished from castor oil, in that castor oil contains esterified fatty acids (mostly as glycerides, and more specifically as triricinoleic glycerides) and has no detectable amount of free ricinoleic acid. As triricinoleic glycerides have chemically distinct characteristics and structure from free ricinoleic acid, it is contemplated that the solubility and/or stability of fulvestrant in triricinoleic glycerides as a solvent will be different from solubility and/or stability of fulvestrant in free ricinoleic acid as a solvent. Preferably, the amount of free ricinoleic acid in the composition is at least 30%, preferably at least 40%, more preferably at least 50%, or between 25-75%, preferably between 30-70%, more preferably between 40-65%. For example, suitable quantities of free ricinoleic acid in contemplated formulations (e.g., for injection) will be between 25-35%, or between 35-45%, or between 45-55%, or between 55-65%, or between 65-75%, or between 20-40%, or between 30-60%, or between 40-75%. Unless noted otherwise, all percentages are (w/v).

With regard to the hydrolyzed castor oil, the same considerations apply. In that context, it should be appreciated that the hydrolyzed castor oil may be fully hydrolyzed, or only partially hydrolyzed, so long as at least 10%, more typically at least 25%, even more typically at least 50%, and most typically at least 75% of all ester bonds between glycerol and ricinoleic acid are hydrolyzed. Viewed from a different perspective, hydrolyzed castor oil may provide at least 10%, or at least 20%, or at least 30%, or at least 40%, or at least 50%, or at least 60%, or at least 70% free ricinoleic acid in the liquid solvent. Moreover, hydrolyzed castor oil may be partially purified, and all forms are deemed suitable for use herein. Moreover, the manner of saponification of the castor oil is not deemed critical and will include enzymatic hydrolysis using lipases as well as chemical hydrolysis/transesterification. Moreover, hydrolyzed castor oil is also commercially available from various sources.

Still further, it should be noted that use of ricinoleic acid or hydrolyzed castor oil has a significant impact on viscosity, and formulations comprising ricinoleic acid or hydrolyzed castor oil will have a viscosity that is typically well below 120 cp, or below 100 cP, or below 80 cP, or below 70 cP, or below 60 cP, or even below 50 cP, which will result in injectable formulations that produce substantially less patient discomfort. In addition, where hydrolyzed castor oil is used, adverse injection site reactions (especially inflammation) is significantly reduced.

In further investigations, the inventors observed that free ricinoleic acid alone may not be an effective solvent for fulvestrant where the fulvestrant concentrations of more than 20 mg/ml are desired to reduce the volume of injection (see exemplary solubility data in Table 1 below). Indeed, fulvestrant solubility in free ricinoleic acid alone is not significantly different from that of castor oil (26 mg/ml v. 21.2 mg/ml). To circumvent at least some of the issues associated with solubility, the inventors further found that the solubility of fulvestrant in free ricinoleic acid solvent can be substantially increased when the solvent includes one or more co-solvents, and especially an alcohol, for example, ethanol, benzyl alcohol, or preferably both. In one preferred embodiment, the concentration of ethanol is less than 30%, preferably less than 20% (w/v), and more preferably less than 15% (w/v), or between 5-30% (w/v), preferably between 5-20% (w/v), and more preferably between 5-15% (w/v). Thus, suitable ethanol concentrations will be between 5-10% (w/v), or between 10-20% (w/v), or between 15-30% (w/v). With respect to benzyl alcohol, it is preferred that the concentration of benzyl alcohol is less than 30% (w/v), preferably less than 20% (w/v), and more preferably less than 15% (w/v), or between 5-30% (w/v), preferably between 5-20% (w/v), and more preferably between 5-15% (w/v). In some embodiments, the concentration of ethanol and benzyl alcohol in the formulation is substantially same (e.g., 10-15% ethanol and 10-15% benzyl alcohol, etc.). However, it is also contemplated that the concentration of ethanol and benzyl alcohol in the formulation can be different at least for 5% or more (e.g., 10% ethanol and 15% benzyl alcohol, etc.). However, it is generally preferred the amount of total alcoholic compounds (ethanol and benzyl alcohol) is no more than 40%, preferably no more than 30%, more preferably no more than 25% (w/v) in the entire solution.

In some embodiments, the monohydroxylated fatty acids (e.g., free ricinoleic acid) in the solvent can be substituted at least in part with other lipid solvents such as castor oil and/or oleic acid. Moreover, various detergents or surfactants can be added to increase solubility as is shown in more detail below. Here, some formulations including these lipid solvents, in combination with ethanol and/or benzyl alcohol, can solubilize fulvestrant at concentrations higher than 60 mg/ml. However, most of such formulations that are capable of solubilizing fulvestrant at a higher concentration (e.g., higher than 60 mg/ml) require higher concentration of alcohols (e.g., higher than 20, 25, 30%, etc.), which may cause instability of the formulation leading to precipitation of fulvestrant over time.

In this context it must be appreciated that while fulvestrant can be solubilized to at least some degree in various solvents, such solubilization is often temporary and fulvestrant will precipitate out of solution within several hours or days from the solvent under ambient temperature in vitro, or after injection in vivo or in a simulated injection model in vitro using a gelatin block. As described in more detail below, the inventors have formulated various compositions that were capable of solubilizing fulvestrant in a concentration of 90 mg/ml or higher and that had excellent results in the gelatin-gel precipitation test.

For example, and as is shown in more detail below, among 10 different formulations 8 formulations showed precipitations of fulvestrant (tested by gelatin test) in 24 hours, while one formulation showed precipitation in 48 hours after the formulation was added to the assay. Notably, the inventors found that one formulation showed no precipitation and had 40% ricinoleic acid rather than other types of lipid solvents (e.g., castor oil, oleic acid, lesquerolic acid, etc.), indicating that the ricinoleic acid (or hydrolyzed castor oil) in the formulation may play a critical role in preventing precipitation of the formulation, and may not be effectively substituted with other types of lipid solvents. Further improvements were achieved by use of various co-solvents, including oleic acid and/or benzyl benzoate. Here, concentrations of the oleic acid and benzyl benzoate preferably do not exceed 25% (w/v), 20%, and more preferably 15% each, and in combination, do not exceed 40%, and more preferably 30% of entire formulation. Thus, the concentration of oleic acid can be between 1-25%, preferably between 5-20%, more preferably between 10-15%, and the concentration of benzyl benzoate can be between 1-20%, and preferably between 5-15%.

Therefore, the inventors contemplate that the solvents including ricinoleic acid and/or hydrolyzed castor oil (and ethanol and/or benzyl alcohol) can increase the concentration of fulvestrant in the formulation to higher than 60 mg/ml, or higher than 70 mg/ml, or higher than 80 mg/ml, or higher than 90 mg/ml, or even higher than 100 mg/ml without generating any significant side products or impurities, while at the same time allowing fulvestrant to remain dissolved in solution over a period of at least two days, or at least three days, or at least five days, or at least one week, or at least two weeks, or at least one month, or at least 2 months.

Thus, the solvent allows to form a fulvestrant injection formulation in a volume of less than 10 ml, preferably less than 7 ml, more preferably about 5 ml or less (with 500 mg/injection dose) such that the number of injections or injection volume can be significantly reduced compared to currently available fulvestrant injection formulations using castor oil as a solvent. Also, such solvents can increase the shelf-life of the fulvestrant solutions in a liquid form without producing any precipitation in vitro in gelatin block test. Further, such solvents can be used to generate a fulvestrant formulation that can be administered to the patient safely without any significant injection site reactions, which mainly results from the large volume of the formulation and the precipitation of fulvestrant after the injection. Consequently, and among other contemplated compositions, contemplated formulations include those that include 10% (+/−3%) ethanol, 10% (+/−3%) benzyl alcohol, 15% (+/−5%) oleic acid, optionally up to 15% (+/−5%) benzyl benzoate with the remainder of the solvent (q.s.) ricinoleic acid and/or hydrolyzed castor oil.

In addition, the inventors further contemplate that the solvents including ricinoleic acid and hydrolyzed castor oil (and ethanol and/or benzyl alcohol) can be used to solubilize lipophilic compounds other than fulvestrant. As used herein, the lipophilic compounds refer any compounds that have partition coefficients of at least 2, preferably at least 3 in a 1-octanol/water system. For example, suitable lipophilic compounds contemplated herein include various steroid drugs, various statins, cyclosporine, ketoprofen, itroconazole, carvedilol, etc. As will be readily appreciated, the concentration or ratio of the ricinoleic acid (and ethanol and/or benzyl alcohol) in the solvent may vary depending on the type of lipophilic compounds and the desired doses of those compounds. Viewed from a different perspective, the inventors therefore contemplate the use of ricinoleic acid and/or hydrolyzed castor oil for making a liquid pharmaceutical preparation (e.g., an injectable solution, an ophthalmic solution, an ingestible solution such as a syrup or an elixir, an inhalable aerosol solution, etc.)

### EXAMPLES

Solubility: Based on solubility studies for fulvestrant, the inventors contemplate that free ricinoleic acid per se may not be an entirely effective solvent for fulvestrant where the concentration of more than 60 mg/ml is desired. Table 1 shows solubility of fulvestrant in various different solvents. As can be seen from the Table, the fulvestrant solubility in free ricinoleic acid alone is not significantly different from that of castor oil (26 mg/ml v. 21.2 mg/ml).

TABLE 1

| | | Solubility |
|---|---|---|
| No | Vehicles | (mg/mL) |
| 1 | Castor Oil | 21.2 |
| 2 | Oleic Acid | 4.8 |
| 3 | Ricinoleic acid | 26 |
| 4 | Propylene Glycol | 39.1 |
| 5 | PEG 300 | 28.3 |
| 6 | PEG 400 | 24.8 |
| 7 | Benzyl Benzoate | 3.9 |
| 8 | Cottonseed Oil | 0.5 |
| 9 | Ethanol | 445.3 |
| 10 | Benzyl Alcohol | 268.8 |
| 11 | Soyabean oil | 0.5 |
| 12 | Olive oil | 0.4 |
| 13 | N,N-dimethylacetamide (DCAM) | 305.9 |
| 14 | Dimethylsulfoxide (DMSO) | 319.6 |
| 15 | Propanoic Acid | 319.0 |
| 16 | Hexanoic Acid | 266.0 |
| 17 | Heptanoic Acid | 154.5 |
| 18 | Octanoic Acid | 92.2 |
| 19 | Butyric Acid | 318.0 |
| 20 | Sesame Oil | 0.5 |
| 21 | Cremophor RH40 | 47.25 |

To increase solubility, various other solvents, co-solvents, and surfactants were tested. For example, in some embodiments the monohydroxylated fatty acids (e.g., free ricinoleic acid) in the solvent were substituted with other lipid solvents such as castor oil or oleic acid. Table 2 shows exemplary results for saturation solubility of fulvestrant in different solutions with combinations of solvents/detergents. Here, some formulations including lipid solvents (especially in combination with ethanol and/or benzyl alcohol) can solubilize fulvestrant at a concentration higher than 60 mg/ml. Yet, most of such formulations capable of solubilizing fulvestrant at a higher concentration (e.g., higher than 60 mg/ml) require higher concentration of alcohols (e.g., higher than 30%, etc.), which may cause instability of the formulation (e.g., precipitation of fulvestrant over time, etc.). Moreover, some solvents also required substantial quantities of emulsifier (e.g., Cremophor™ (hydrogenated castor oil reacted with ethylene oxide)), which is not desirable as an ingredient for injection at the tested concentrations. Indeed, it is generally preferred that the fulvestrant compositions and solutions for injection are substantially free (i.e., less than 1% w/v, or less than 0.1% w/v, or less than 0.01% w/v) from surfactants, detergents, and/or emulsifiers.

TABLE 2

| | | | | | | Solubility |
|---|---|---|---|---|---|---|
| No | Components (% w/v) | | | | | (mg/mL) |
| 1 | 99% Oleic Acid | 1% Poly. 20 | — | — | — | 4.08 |
| 2 | 99% Castor Oil | 1% Poly. 20 | — | — | — | 23.16 |
| 3 | 50% Castor Oil | 50% Oleic | — | — | — | 20.00 |
| | | Acid | | | | |
| 4 | 25% Oleic Acid | 75% Castor Oil | | | — | 18.20 |
| 5 | 75% Oleic Acid | 25% Castor | — | — | — | 15.50 |
| | | Oil | | | | |
| 6 | 49.5% Oleic Acid | 49.5% Castor | 1% Polysorbate | — | — | 22.80 |
| | | Oil | 20 | | | |
| 7 | 74.5% Oleic Acid | 24.5% Castor | 1% Polysorbate | — | — | 15.70 |
| | | Oil | 20 | | | |
| 8 | 50% Oleic Acid | 25% Ethanol | 25% Cremophor | — | — | 130.40 |
| | | | 40 | | | |
| 9 | 50% Castor Oil | 25% Ethanol | 25% Cremophor | — | — | 133.60 |
| | | | 40 | | | |
| 10 | 75% Oleic Acid | 12.5% Ethanol | 12.5% | — | — | 113.40 |
| | | | Cremophor RH | | | |
| | | | 40 | | | |
| 11 | 12.5% Ethanol | 75% Castor Oil | 12.5% | — | — | 128.30 |
| | | | Cremophor RH | | | |
| | | | 40 | | | |
| 12 | 50% Castor Oil | 10% Lipoid E | 40% Ethanol | — | — | 49.28 |
| | | 80 | | | | |
| 13 | 15% Ethanol | 15% Castor Oil | 15% Benzyl | 10% Lipoid E 80 | — | 45.95 |
| | | | Alcohol | | | |
| 14 | 60% Castor Oil | 18% BA | 13% Ethanol | 9% Lipoid E 80 | — | 63.00 |
| 15 | 85% Castor Oil | 12% BA | 3% HP-B-CD | — | — | 17.19 |
| 16 | 70% Castor Oil | 20% Oleic Acid | 5% BA | 5% Ethanol | — | 49.90 |
| 17 | 10% Ethanol | 65% Castor Oil | 20% Oleic Acid | 5% Benzyl Alcohol | — | 68.76 |
| 18 | 5% Ethanol | 65% Castor Oil | 20% Oleic Acid | 10% Benzyl Alcohol | | 39.48 |
| 19 | 22.5% Castor Oil | 67.5% Oleic | 5% BA | 5% Ethanol | — | 47.32 |
| | | Acid | | | | |
| 20 | 45% Oleic Acid | 45% Castor | 5% Ethanol | 5% Benzyl Alcohol | — | 43.68 |
| | | Oil | | | | |
| 21 | 5% Cremophor | 5% Ethanol | 5% Benzyl | 85% Castor Oil | — | 24.36 |
| | EL 35 | | Alcohol | | | |
| 22 | 5% HS 15 | 5% Ethanol | 5% Benzyl | 85% Castor Oil | — | 39.24 |
| | | | Alcohol | | | |
| 23 | 5% Cremophor | 5% Ethanol | 5% Benzyl | 85% Castor Oil | — | 38.04 |
| | RH 40 | | Alcohol | | | |
| 24 | 44.5% Oleic Acid | 44.5% Castor | 5% Ethanol | 5% Benzyl Alcohol | 1% Poly. | 51.90 |
| | | Oil | | | 20 | |
| 25 | 67% Oleic Acid | 22% Castor | 5% Ethanol | 5% Benzyl Alcohol | 1% Poly. | 41.00 |
| | | Oil | | | 20 | |

To reduce or even entirely avoid use of surfactants, detergents, and/or emulsifiers as well as reduce concentration of alcohols, ricinoleic acid (and hydrolyzed castor oil, data not shown) was tested as a solvent or component in a solvent/co-solvent system. Notably, various formulations that are capable of solubilizing fulvestrant in a concentration of 90 mg/ml or higher were obtained and tested with gelatin-gel precipitation test to evaluate the stability of the formulation. Table 3 shows exemplary results for the simulated in vitro stability of the fulvestrant formulations at targeted solubility as indicated using a gelatin block assay (*F: No precipitation was observed at 24 hrs.; however precipitation was observed post 48 hrs of injection).

TABLE 3

| | | | | | | | | | | Gelatin | |
|---|---|---|---|---|---|---|---|---|---|---|---|
| | | Benzyl | Castor | Oleic | PEG | Ricinoleic | Lesquerolic | Benzyl | Solubility | Test | Time to |
| ID | Ethanol | Alcohol | Oil | Acid | 300 | Acid | Oil | Benzoate | (mg/mL) | (Precip.) | precip. |
| A | 10.00% | 10.00% | — | — | — | — | Q.S to | 15.00% | 91.5 | Yes | <24 Hrs |
| | | | | | | | 6 ml | | | | |
| B | 10.00% | 10.00% | Q.S to | — | — | — | — | 15.00% | 95.8 | Yes | <24 Hrs |
| | | | 6 ml | | | | | | | | |
| C | 10.00% | 10.00% | Q.S to | 40.00% | — | — | — | — | 112.7 | Yes | <24 Hrs |
| | | | 6 ml | | | | | | | | |
| D | 10.00% | 10.00% | Q.S to | — | — | 40.00% | — | — | 101 | No | No |
| | | | 6 ml | | | | | | | | precipitation |
| E | 10.00% | 10.00% | Q.S to | 40.00% | 5.00% | — | — | — | 102.9 | Yes | <24 Hrs |
| | | | 6 ml | | | | | | | | |
| F | 10.00% | 10.00% | Q.S to | 32.50% | — | — | — | 15.00% | 100.1 | Yes* | <48 Hrs |
| | | | 6 ml | | | | | | | | |
| G | 10.00% | 10.00% | Q.S to | — | — | 32.50% | — | 15.00% | 94.8 | Yes | <24 Hrs |
| | | | 6 ml | | | | | | | | |
| H | 10.00% | 10.00% | — | 40.00% | — | — | Q.S to | — | 101.6 | Yes | <24 Hrs |
| | | | | | | | 6 ml | | | | |
| I | 10.00% | 10.00% | — | 40.00% | — | Q.S to | — | — | 95.6 | Yes | <24 Hrs |
| | | | | | | 6 ml | | | | | |
| L | 10.00% | 10.00% | Q.S to | — | — | — | — | 15.00% | 50.15 | Yes | <24 Hrs |
| | | | 6 ml | | | | | | | | |

While ricinoleic acid provided increased solubility, stability was not desirable in most cases. Therefore, the inventor tested further solvent systems that included various alcohols and/or benzyl benzoate. In most of the tested systems, concentrations of the oleic acid and benzyl benzoate did not exceed 25% (w/v), 20%, and more preferably 15% each, and in combination, did not exceed 40%, and more preferably 30% of the entire formulation. In these and other experiments, the concentration of oleic acid was between 1-25%, preferably between 5-20%, more preferably between 10-15%, and the concentration of benzyl benzoate can be between 1-20%, and preferably between 5-15%. Table 4 and Table 5 show exemplary formulations along with the concentrations of the components that can provide desirable solubility, viscosity, stability (lack of precipitation) for fulvestrant solutions at high fulvestrant concentrations. Table 4 depicts exemplary viscosity and stability results, while Table 5 depicts exemplary additional results for viscosity and stability (PPT: Precipitation).

TABLE 4

| | | | | | | | | | 24 | 48 | 72 |
|---|---|---|---|---|---|---|---|---|---|---|---|
| | | Benzyl | Castor | Oleic | Ricinoleic | Benzyl | | | hr | hr | hr |
| | Ethanol | Alcohol | Oil | Acid | acid | Benzoate | Fulvestrant | Viscosity | Gelatin | | |
| ID | (% w/v) | (% w/v) | (% w/v) | (% w/v) | (% w/v) | (% w/v) | (mg/mL) | (cP) | Block Study | | |
| RD035-094 A | 10 | 10 | — | — | QS (S) | — | 100 | 150.00 | √ | √ | √ |
| RD035-094 B | 10 | 10 | — | — | QS (S) | 15 | 100 | 65.71 | √ | √ | √ |
| RD035-094 C | 10 | 10 | — | — | QS (P) | — | 100 | 125.80 | √ | √ | √ |
| RD035-094 D | 10 | 10 | — | — | QS (P) | 15 | 100 | 58.42 | √ | √ | √ |
| RD035-094 E | 10 | 10 | — | QS | — | — | 100 | ND | X | X | X |
| RD035-094 F | 10 | 10 | — | QS | — | 15 | 100 | ND | √ | √ | √ |
| RD035-094 G | 10 | 10 | — | 10 | QS (P) | — | 100 | 90.70 | √ | √ | √ |
| RD035-094 H | 10 | 10 | — | 10 | QS (P) | 15 | 100 | 54.98 | √ | √ | √ |
| RD035-094 I | 10 | 10 | QS | 40 | — | — | 100 | ND | X | X | X |
| RD035-094 J | 10 | 10 | QS | 40 | — | 15 | 100 | ND | X | X | X |
| RLD | 10 | 10 | QS | — | — | 15 | 100 | 72.86 | √ | √ | √ |

TABLE 5

| | | Benzyl | Ricinoleic | Oleic | Benzyl | | | | | Fulv. | Sat. |
|---|---|---|---|---|---|---|---|---|---|---|---|
| | Ethanol | Alcohol | acid | acid | Benzoate | Gelatin Block Study | | | Viscosity | Solub. | Solub. |
| ID | (% w/v) | (% w/v) | (% w/v) | (% w/v) | (% w/v) | 24 hr | 48 hr | 72 hr | (cP) | (mg/mL) | (mg/mL) |
| 1 | 10 | 10 | 65 | 00 | 15 | Clear | Clear | Clear | 68.32 | 102.0 | 162.7 |
| 2 | 10 | 20 | 40 | 15 | 15 | Clear | PPT | PPT | 25.96 | 93.1 | 148.1 |
| 3 | 10 | 20 | 65 | 05 | 00 | Clear | Clear | Clear | 76.13 | 102.0 | 164.3 |
| 4 | 10 | 17 | 65 | 00 | 08 | Clear | Clear | Clear | 69.91 | 98.4 | 158.5 |
| 5 | 10 | 10 | 65 | 15 | 00 | Clear | Clear | Clear | 73.06 | 88.8 | 144.2 |
| 6 | 10 | 20 | 50 | 14 | 06 | Clear | Clear | Clear | 39.96 | 90.9 | 148.3 |
| 7 | 10 | 10 | 40 | 29 | 11 | Clear | PPT | PPT | 35.77 | 91.1 | 145.9 |
| 8 | 10 | 10 | 55 | 18 | 08 | Clear | PPT | PPT | 45.47 | 103.6 | 165.8 |
| 9 | 10 | 20 | 40 | 30 | 00 | PPT | PPT | PPT | 33.11 | 93.3 | 146.5 |
| 10 | 10 | 10 | 53 | 13 | 15 | Clear | Clear | Clear | 43.43 | 98.6 | 161.9 |
| 11 | 10 | 15 | 54 | 21 | 00 | Clear | Clear | Clear | 52.12 | 93.0 | 153.9 |
| 12 | 10 | 15 | 40 | 35 | 00 | Clear | PPT | PPT | 45.88 | 89.4 | 148.6 |
| 13 | 10 | 10 | 45 | 35 | 00 | Clear | PPT | PPT | 38.32 | 82.9 | 140.3 |
| 14 | 10 | 20 | 55 | 00 | 15 | Clear | Clear | Clear | 44.66 | 87.5 | 137.8 |
| 15 | 15 | 15 | 51 | 13 | 07 | Clear | Clear | Clear | 29.28 | 93.5 | 157.2 |
| 16 | 15 | 15 | 51 | 13 | 07 | Clear | Clear | Clear | 30.35 | 95.3 | 149.6 |
| 17 | 15 | 20 | 40 | 25 | 00 | PPT | PPT | PPT | 21.46 | 95.6 | 159.1 |
| 18 | 18 | 10 | 40 | 33 | 00 | Clear | PPT | PPT | 21.66 | 96.8 | 156.6 |
| 19 | 20 | 10 | 65 | 00 | 05 | Clear | Clear | Clear | 30.35 | 102.7 | 166.5 |
| 20 | 20 | 20 | 45 | 00 | 15 | PPT | PPT | PPT | 15.33 | 93.4 | 154.0 |
| 21 | 20 | 15 | 40 | 10 | 15 | Clear | PPT | PPT | 15.33 | 93.1 | 145.6 |
| 22 | 20 | 10 | 40 | 21 | 09 | Clear | PPT | PPT | 17.27 | 94.2 | 151.0 |
| 23 | 20 | 15 | 40 | 25 | 00 | PPT | PPT | PPT | 18.09 | 95.8 | 157.2 |
| 24 | 20 | 16 | 56 | 00 | 09 | Clear | PPT | PPT | 21.26 | 94.3 | 152.3 |
| 25 | 20 | 10 | 55 | 00 | 15 | Clear | PPT | PPT | 35.56 | 95.8 | 151.9 |
| 26 | 20 | 20 | 40 | 14 | 06 | Clear | PPT | PPT | 12.23 | 103.5 | 166.7 |
| 27 | 20 | 10 | 53 | 18 | 00 | Clear | Clear | PPT | 24.22 | 96.8 | 161.2 |
| 28 | 20 | 20 | 60 | 00 | 00 | PPT | PPT | PPT | 23.71 | 97.2 | 163.7 |
| 29 | 20 | 10 | 65 | 05 | 00 | Clear | Clear | PPT | 30.96 | 98.8 | 155.3 |
| 30 | 20 | 20 | 48 | 12 | 00 | Clear | PPT | PPT | 24.53 | 93.3 | 150.0 |

Toxicology and Pharmacokinetics of selected formulations was tested in selected formulations in female rats: The pharmacokinetics and injection site pathology of fulvestrant formulations when administered as a single intramuscular injection were evaluated in female Hsd:Sprague Dawley®™ SD®™ rats. Groups of six rats were administered single intramuscular injections of RLD (control article) of each of four formulations as indicated below in Tables 8-11 in the left biceps femoris. The corresponding vehicle for each formulation was injected into the right biceps femoris of each animal and served as the vehicle control for each formulation. Animals were observed for clinical signs of the drug's effects. Blood samples for bioanalytical analysis were collected over fourteen days and the plasma concentration of fulvestrant was analyzed for the pharmacokinetic profile of each formulation. The animals were euthanized for postmortem examinations, and the tissue harvested from the injection sites and evaluated microscopically. The study design was as shown in Table 6 below.

TABLE 6

| | | Dosage Left Leg | | Dosage Right Leg | |
|---|---|---|---|---|---|
| | | Concentration | Dose | Concentration | Dose |
| Study | | Fluvestrant | Volume | Fluvestrant | Volume |
| Group | Test Article | (mg/mL) | (mL/kg) | (mg/mL) | (mL/kg) |
| 1 | RLD | 50 | 80 | 0 | 80 |
| 2 | Formulation 1 | 100 | 40 | 0 | 40 |
| 3 | Formulation 2 | 100 | 40 | 0 | 40 |
| 4 | Formulation 3 | 100 | 40 | 0 | 40 |
| 5 | Formulation 4 | 100 | 40 | 0 | 40 |

Pharmacokinetic results are presented in Table 7 below. The formulations were considered to be equivalent to the RLD (Reference Listed Drug) if the Cₘₐₓ and AUC₍₀₋ₜ₎ of each formulation were within 80%-125% of the RLD (Cₘₐₓ=69.90 to 109.25 ng/mL and AUC₍₀₋ₜ₎=15,488 to 24,200 ng·h/mL).

In each case the formulations 1-4 met the requirement and were thereby considered equivalent to the RLD for Cₘₐₓ and exposure (AUC) (see Table 7). Further based on the MRT (Mean Resident Time), which reflects the amount of time the drug remains at the site of action, the test results were similar for the RLD and formulations 1-4 (150 h, 150 h, 136 h, 144 h and 146 h respectively).

In contrast the Clearance rate (L/h/kg) was reduced in the formulations 1-4 compared to the RLD (RLD=4180 and the formulations 1-4=1990, 2580, 2410, 2590, respectively) indicating that the formulations cleared the plasma at a slower rate that the RLD.

TABLE 7

| | | | | | Equivalence |
|---|---|---|---|---|---|
| | | | | | Compared |
| | | | | | to the RLD¹ |
| | | | | | for |
| Dose | | | | | AUC (80% |
| Group | Test Article | Endpoint | Mean | SD | to 125%) |
| 1 | RLD | Cₘₐₓ (ng/mL) | 87.4 | 16.58 | 69.90 to 109.25 |
| | | AUC(0 − t) | 19,360 | 1130 | 15,488 to 24,200 |
| | | (ng•h/mL) | | | |
| | | MRT (h) | 150 | 6.53 | |
| | | Cl (L/h/kg) | 4180 | 501 | |
| 2 | Formulation 1 | Cₘₐₓ (ng/mL) | 106 | 23.6 | + |
| | | AUC(0 − t) | 20,600 | 3,140 | + |
| | | (ng•h/mL) | | | |
| | | MRT (h) | 150 | 7.76 | |
| | | Cl (L/h/kg) | 1990 | 348 | |
| 3 | Formulation 2 | Cₘₐₓ (ng/mL) | 83.1 | 23.3 | + |
| | | AUC(0 − t) | 16,500 | 4,130 | + |
| | | (ng•h/mL) | | | |
| | | MRT (h) | 136 | 10.1 | |
| | | Cl (L/h/kg) | 2580 | 729 | |
| 4 | Formulation 3 | Cₘₐₓ (ng/mL) | 88.9 | 22.2 | + |
| | | AUC(0 − t) | 18,100 | 5,770 | + |
| | | (ng•h/mL) | | | |
| | | MRT (h) | 144 | 7.97 | |
| | | Cl (L/h/kg) | 2410 | 747 | |
| 5 | Formulation 4 | Cₘₐₓ (ng/mL) | 75.2 | 18.8 | + |
| | | AUC(0 − t) | 15,800 | 2,470 | + |
| | | (ng•h/mL) | | | |
| | | MRT (h) | 146 | 9.40 | |
| | | Cl (L/h/kg) | 2590 | 410 | |

¹Equivalence was considered a value within a range of 80 to 125% the mean of the Cₘₐₓ or AUC for the RLD.
A ‘+’ indicates it was equivalent and a ‘−’ not equivalent. The results of this study indicate that all formulation met the criteria and were equivalent.

Tables 8-11 show the compositions (Formulation 1-4) of the injection compositions used in the above experiments.

TABLE 8

| | | | Batch Quantity | |
|---|---|---|---|---|
| Ingredient | Composition | | (10 mL) | |
| Fulvestrant | 100 | mg/mL | 1 | gram |
| Ethanol | 10% | w/v | 1 | gram |
| Benzyl Alcohol | 10% | w/v | 1 | gram |
| Benzyl Benzoate | 15% | w/v | 1.5 | gram |
| Ricinoleic Acid | q.s. 1 | mL | q.s. 10 | mL |

TABLE 9

| | | | Batch Quantity | |
|---|---|---|---|---|
| Ingredient | Composition | | (10 mL) | |
| Fulvestrant | 100 | mg/mL | 1 | gram |
| Ethanol | 10% | w/v | 1 | gram |
| Benzyl Alcohol | 10% | w/v | 1 | gram |
| Oleic Acid | 15% | w/v | 1.5 | gram |
| Ricinoleic Acid | q.s. 1 | mL | q.s. 10 | mL |

TABLE 10

| | | | Batch Quantity | |
|---|---|---|---|---|
| Ingredient | Composition | | (10 mL) | |
| Fulvestrant | 100 | mg/mL | 1 | gram |
| Ethanol | 10% | w/v | 1 | gram |
| Benzyl Alcohol | 10% | w/v | 1 | gram |
| Oleic Acid | 7.5% | w/v | 0.75 | gram |
| Benzyl Benzoate | 7.5% | w/v | 0.75 | gram |
| Ricinoleic Acid | q.s. 1 | mL | q.s. 10 | mL |

TABLE 11

| | | | Batch Quantity | |
|---|---|---|---|---|
| Ingredient | Composition | | (10 mL) | |
| Fulvestrant | 100 | mg/mL | 1 | gram |
| Ethanol | 20% | w/v | 2 | gram |
| Benzyl Alcohol | 10% | w/v | 1 | gram |
| Benzyl Benzoate | 15% | w/v | 1.5 | gram |
| Oleic Acid | 15% | w/v | 1.5 | gram |
| Ricinoleic Acid | q.s. 1 | mL | q.s. 10 | mL |

Additional fulvestrant compositions are shown in Tables 12-14 where concentrations of fulvestrant in the formulations are between 60-90 mg/ml. In these and all of the above formulations, ricinoleic acid can be replaced by hydrolyzed castor oil.

TABLE 12

| S. No. | Ingredient | Composition | |
|---|---|---|---|
| 1 | Fulvestrant | 60 | mg/mL |
| 2 | Ethanol | 10% | w/v |
| 3 | Benzyl Alcohol | 10% | w/v |
| 4 | Benzyl Benzoate | 15% | w/v |
| 5 | Ricinoleic Acid | q.s. 1 | mL |

TABLE 13

| S. No. | Ingredient | Composition | |
|---|---|---|---|
| 1 | Fulvestrant | 75 | mg/mL |
| 2 | Ethanol | 10% | w/v |
| 3 | Benzyl Alcohol | 10% | w/v |
| 4 | Oleic Acid | 15% | w/v |
| 5 | Ricinoleic Acid | q.s. 1 | mL |

TABLE 14

| S. No. | Ingredient | Composition | |
|---|---|---|---|
| 1 | Fulvestrant | 90 | mg/mL |
| 2 | Ethanol | 10% | w/v |
| 3 | Benzyl Alcohol | 10% | w/v |
| 4 | Oleic Acid | 7.5% | w/v |
| 5 | Benzyl Benzoate | 7.5% | w/v |
| 6 | Ricinoleic Acid | q.s. 1 | mL |

In yet further experiments using ricinoleic acid based solvent systems, the inventors discovered various alternative formulations with high saturation solubility (SS), solubility suitable for use in contemplated compositions, and pharmaceutically acceptable components at pharmaceutically acceptable concentrations

As used herein, the term “provide” or “providing” refers to and includes any acts of manufacturing, generating, placing, enabling to use, or making ready to use. As used herein, the term “administering” refers to both direct and indirect administration of the formulation. Direct administration of pharmaceutical compositions contemplated herein is typically performed by a health care professional (e.g., physician, nurse, etc.), while indirect administration includes a step of providing or making available the pharmaceutical compositions to the health care professional for direct administration (e.g., via injection, etc.).

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. An injectable liquid pharmaceutical composition, comprising:
fulvestrant dissolved in a liquid solvent at a concentration of at least 60 mg/ml, wherein the liquid solvent is a pharmaceutically acceptable carrier for injection and comprises free ricinoleic acid.

2. The composition of claim 1, wherein the liquid solvent comprises a free ricinoleic acid, and at least one of ethanol and benzyl alcohol.

3. The composition of claim 2, wherein the free ricinoleic acid is present at a concentration of at least 50% w/v.

4. The composition of claim 2, wherein the ethanol is present at a concentration of between 5-25% w/v.

5. The composition of claim 2, wherein the benzyl alcohol is present at a concentration of between 5-25% w/v.

6. The composition of claim 1, wherein the fulvestrant is dissolved in the liquid solvent at a concentration of at least 80 mg/ml.

7. The composition of claim 1, wherein the fulvestrant is dissolved in the liquid solvent at a concentration of at least 80 mg/ml with a viscosity of less than 100 cP.

8. The composition of claim 1, wherein a solubility of the fulvestrant in the liquid solvent is maintained at least for 48 hours within an in vitro precipitation model.

9. The composition of claim 1, further comprising an oleic acid, wherein the oleic acid is present at a concentration less than 25%, and/or further comprising a benzyl benzoate, wherein the benzyl benzoate is present at a concentration at or less than 15%.

10. The composition of claim 1, wherein the liquid solvent comprises ethanol, benzyl alcohol, oleic acid, and ricinoleic acid, and optionally further comprises benzyl benzoate.

11. The composition of claim 10, wherein the liquid solvent comprises 10% ethanol, 10% benzyl alcohol, 15% oleic acid, and q.s. ricinoleic acid, and optionally wherein a single dose of the injectable liquid pharmaceutical composition is formulated in a volume equal to or less than 5 ml.

12-33. (canceled)

34. An injectable liquid pharmaceutical composition, comprising:
fulvestrant dissolved in a liquid solvent at a concentration of at least 60 mg/ml, wherein the liquid solvent comprises hydrolyzed castor oil, wherein the hydrolyzed castor oil provides at least 10% free ricinoleic acid in the liquid solvent.

35. The composition of claim 34, wherein the liquid solvent further comprises ethanol and/or benzyl alcohol.

36. The composition of claim 34, wherein the hydrolyzed castor oil is present at a concentration of at least 50% w/v.

37. The composition of claim 35, wherein the ethanol is present at a concentration of between 5-25% w/v.

38. The composition of claim 35, wherein the benzyl alcohol is present at a concentration of between 5-25% w/v.

39. The composition of claim 34, wherein the fulvestrant is dissolved in the liquid solvent at a concentration of at least 80 mg/ml.

40. The composition of claim 34, wherein the fulvestrant is dissolved in the liquid solvent at a concentration of at least 80 mg/ml with a viscosity of less than 100 cP.

41. The composition of claim 34, wherein solubility of the fulvestrant in the liquid solvent is maintained at least for 48 hours within an in vitro precipitation model.

42. The composition of claim 34, further comprising oleic acid, wherein the oleic acid is present at a concentration less than 25%, and/or further comprising benzyl benzoate, wherein the benzyl benzoate is present at a concentration at or less than 15%.

43. The composition of claim 34, wherein the liquid solvent comprises ethanol, benzyl alcohol, oleic acid, and optionally further comprises benzyl benzoate.

44. The composition of claim 43, wherein the liquid solvent comprises 10% ethanol, 10% benzyl alcohol, 15% oleic acid, and q.s. hydrolyzed castor oil, and optionally wherein a single dose of the injectable liquid pharmaceutical composition is formulated in a volume equal to or less than 5 ml.
